(19) United States
(12) Patent Application Publication
(10) Pub. No.: US 2010/0248321 A1
(43) Pub. Date: Sep. 30, 2010

(54) SURFACTANT AMENDMENTS FOR THE STIMULATION OF BIOGENIC GAS GENERATION IN DEPOSITS OF CARBONACEOUS MATERIALS

(75) Inventors: Jefferey W. Steaffens, Golden, CO (US); Shelley Haveman, Lakewood, CO (US); Mark Finkelstein, Morrison, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LUCA Technologies, Inc., Golden, CO (US)

(21) Appl. No.: 12/413,401

(22) Filed: Mar. 27, 2009

(51) Int. Cl. C12P 5/00

(52) U.S. Cl. 435/166

(57) ABSTRACT

Methods of conditioning a carbonaceous material in a subterranean geologic formation for metabolism into a compound with enhanced hydrogen content by a microorganism consortium are described. The methods may include the steps of accessing the subterranean geologic formation through an access point, and contacting the carbonaceous material with a surfactant. The microorganism consortium can utilize the surfactant as a first nutrient source. The surfactant also increases accessibility of the carbonaceous material as a second nutrient source for the microorganism consortium. The microorganism consortium metabolizes the carbonaceous material into the compound with the enhanced hydrogen content.

### CROSS-REFERENCES TO RELATED APPLICATIONS

NOT APPLICABLE

### BACKGROUND OF THE INVENTION

Economic and environmental pressures are encouraging the use of natural gas as an energy source for heating, electric power generation, and increasingly as a transportation fuel. Natural gas has a higher atomic ratio of hydrogen-to-carbon than oil or coal, resulting in lower quantities of the greenhouse gas carbon dioxide per unit of energy than traditional fossil fuels. Natural gas can also be used as a feedstock for other clean-burning transportation fuels like molecular hydrogen.

Major sources of natural gas come from the same subterranean formations that contain large quantities of liquid and solid carbonaceous materials such as oil fields and coal beds. A significant portion of this natural gas produced is believed come from biogenic sources, such as microorganisms living in the formations that metabolize the carbonaceous material and excrete natural gas (e.g., methane) as a metabolic product. In formations where these microorganisms have been converting the carbonaceous material to natural gas for thousands, or even millions of years, the buildup of biogenically produced natural gas can be measured in the trillions of cubic feet (Tcf).

As these large reserves of natural gas created over many thousands of years are depleted, the natural gas economy faces a similar important question as traditional fossil fuels: When will peak production be reached as the majority of these reserves are recovered? Fortunately, the biogenic processes that originally produced much of this natural gas could still be harnessed to continue producing gas on a globally significant scale. If biogenic processes can be enhanced to convert even a small fraction of the existing carbonaceous material in mature coal beds and oil fields to natural gas, the quantities are enormous. For example, the Powder River Basin in northeastern Wyoming is estimated to contain approximately 1,300 billion short tons of coal. If just 1% of this coal were biogenically converted to natural gas, it could supply the current annual natural gas usage in the United States (i.e., about 23 trillion cubic feet) for four years. There are several mature coal and oil fields estimated to have these quantities of residual carbonaceous material in the United States alone.

One of the challenges faced in the biogenic conversion of these carbonaceous materials to natural gas and other biogenically produced hydrocarbons is making the carbonaceous material accessible to the microorganisms that do the metabolizing. This can be particularly challenging for solid and semi-solid carbonaceous materials. For example, coals are generally composed of large, aromatic macromolecular structures that are difficult for microorganisms to break apart and metabolize. This can slow or stop the biogenic conversion of these materials into natural gas, as well as limit the population growth of the microorganisms trying to utilize them as an energy source. Thus, there is a need to make carbonaceous materials more accessible to the microorganisms so they can metabolize them at a faster rate or with less energy, or both.

Carbonaceous materials also typically include a combination of carbon-containing compounds that can be metabolized to varying extents by the microorganisms. Larger macromolecules (e.g., a large, tightly-packed polyaromatic ring structures) are generally considered to be harder to metabolize than smaller hydrocarbons such as short-chained alkanes and monoaromatic ring compounds. Separating the larger compounds from the smaller compounds, and moving the smaller compounds into contact with the microorganisms may significantly enhance the rate of metabolism of the carbonaceous material. Thus, there is a need to make carbonaceous materials more accessible to the microorganisms by moving the more convertible compounds in the material into contact with the microorganisms.

### BRIEF SUMMARY OF THE INVENTION

Methods are described for providing surfactant compositions to geologic formations of carbonaceous materials in order to increase the biogenic production of natural gas and other useful metabolic products from microorganisms living in the formation. The surfactant compositions are selected to increase the accessibility of the carbonaceous material to the microorganisms. The increased accessibility may result from increased contact between the carbonaceous materials and the microorganisms. It may also result from dissolving and migrating constituents sequestered in the material to areas that are more easily accessible by the microorganisms.

The surfactants themselves may also act as a nutrient source for the microorganisms. They may be converted through the same methanogenic pathways into the same (or similar) metabolic products as the carbonaceous material. Selecting surfactants that act as both a nutrient source and a facilitator of increased accessibility to the carbonaceous material can help a microorganism consortium to grow in proximity to the carbonaceous material: Initially the consortium may grow primarily or exclusively by metabolizing the surfactant. Over time more of the consortium's nutrients come from constituents of the carbonaceous material, which are made available by the action of the surfactant.

Embodiments of the invention include methods of increasing biogenic production of a metabolic product with enhanced hydrogen content. The method may include the steps of accessing a subterranean geologic formation that includes a carbonaceous material, and providing a surfactant containing solution to the geologic formation. The surfactant solution can increase a rate at which the metabolic product is biogenically produced in the geologic formation.

Embodiments of the invention further include methods of conditioning a carbonaceous material in a subterranean geologic formation for metabolism into a compound with enhanced hydrogen content by a microorganism consortium. The methods may include the steps of accessing the subterranean geologic formation through an access point, and contacting the carbonaceous material with a surfactant. The microorganism consortium can utilize the surfactant as a first nutrient source. The surfactant also increases accessibility of the carbonaceous material as a second nutrient source for the microorganism consortium. The microorganism consortium metabolizes the carbonaceous material into the compound with the enhanced hydrogen content.

Embodiments of the invention also include methods of increasing the accessibility of a carbonaceous material in a subterranean geologic formation to a microorganism consortium. The methods may include accessing the subterranean geologic formation, and contacting the carbonaceous material with a surfactant. The surfactant can move a first hydrocarbon from the carbonaceous material into contact with the microorganism consortium. The microorganism consortium can also metabolize the first hydrocarbon into a metabolic product with enhanced hydrogen content compared with the first hydrocarbon species.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

A further understanding of the nature and advantages of the present invention may be realized by reference to the remaining portions of the specification and the drawings wherein like reference numerals are used throughout the several drawings to refer to similar components. In some instances, a sub-label is associated with a reference numeral and follows a hyphen to denote one of multiple similar components. When reference is made to a reference numeral without specification to an existing sub-label, it is intended to refer to all such multiple similar components.

FIG. 1 is a flowchart illustrating methods of applying a surfactant solution to a subterranean geologic formation according to embodiments of the invention;

FIG. 2 is a flowchart illustrating methods of conditioning carbonaceous material for increased methanogenesis with a surfactant according to embodiments of the invention;

FIG. 3 is a flowchart showing methods of conditioning carbonaceous material to a methanogenic microorganism consortium according to embodiments of the invention;

FIG. 4 is a flowchart showing methods of stimulating methanogenesis by providing a microorganism consortium with a surfactant according to embodiments of the invention; and

FIGS. 5A-C show exemplary structures for three types of macromolecules found in coal.

### DETAILED DESCRIPTION OF THE INVENTION

Methods are described for increasing the rate of biogenically produced compounds such as methane by providing surfactant compositions to geologic formations containing carbonaceous material. Surfactants are compounds that are active at the interface between two phases, such as the interface between coal or shale and water. Surfactants tend to accumulate at this interface and can modify its surface tension to allow easier distribution of materials between the phases. This property of surfactants can serve to increase the accessibility of more easily metabolizable components of the carbonaceous material by a microorganism consortium. The increased accessibility may come from transporting these components (typically non-polar hydrocarbons) to polar, aqueous fluid media where the microorganisms reside. It may also come from increasing the penetration and spread of microorganism carrying fluids through the carbonaceous material.

Select surfactants can also act as a food source for at least some populations of microorganisms in the consortium. Simple surfactants may be directly metabolized for energy, while more complex surfactants may include easily separated moieties that can be metabolized. Because surfactants typically concentrate at phase boundaries they can provide a source of food that is localized close to the bulk of the carbonaceous material. This can encourage the growth of the microorganism consortium closer to the carbonaceous material, which may encourage the consortium to rely more on the material as a nutrient source. In some instances, the surfactant may act as a temporary, initial nutrient source that gives the consortium time to adapt to the carbonaceous material as a predominant (or even exclusive) source of food.

Surfactants may also act as an activation, initiation, and catalytic compounds for increasing the production rate of biogenically produced materials such as methane. In this role, the surfactant may be lowering an activation barrier, opening a metabolic pathway, modifying a carbonaceous material, changing the ambient reaction environment, etc., without being rapidly consumed as a nutrient. Thus, the introduction of small quantities or concentrations of the surfactant to the formation can produce much more than stoichiometric quantities of the biogenically produced materials, and/or increase the production rate of these materials for an extended period. In some instances, it can even be the case that smaller quantities and/or more dilute concentrations of an activator surfactant enhance production rates more than the application of larger quantities and/or higher concentrations.

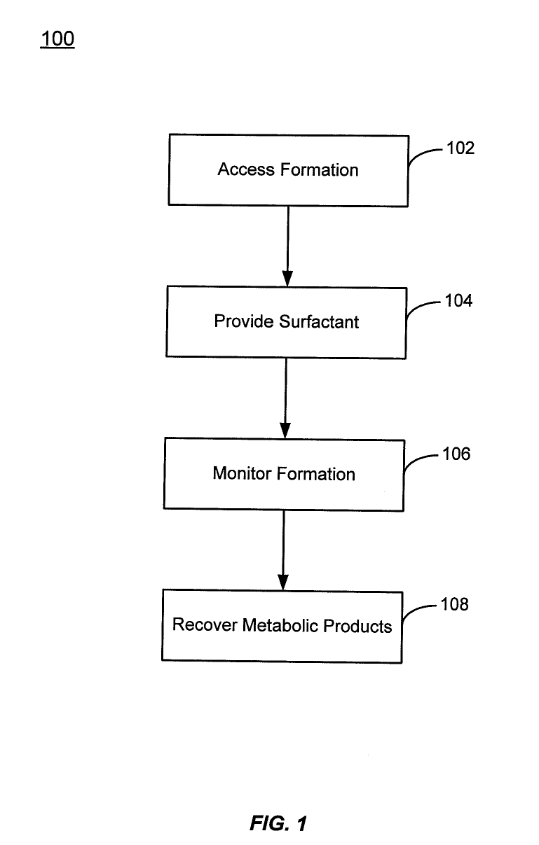

Referring now to FIG. 1, a flowchart illustrating selected steps in a method 100 of applying a surfactant solution to a subterranean geologic formation according to embodiments of the invention is shown. The method 100 includes accessing a subterranean geologic formation that contains carbonaceous material 102. The geologic formation may be a previously explored, carbonaceous material containing formation such as a coal field, oil field, natural gas deposit, or carbonaceous shale deposit, among other formations. In many instances, the formation may be accessed through previously mined or drilled access points used to recover carbonaceous material. For previously unexplored formations, access may involve digging or drilling through a surface layer to access an underlying site containing carbonaceous material.

Once access is gained to the carbonaceous material in the formation, a surfactant may be provided to the material 104. If the surfactant is a liquid at ambient temperature, it may be directly poured, sprayed, injected, etc., into the access point. Alternatively, the surfactant may be combined with additional components of an amendment for stimulating methanogenic activity in the formation. For example, the surfactant may be added to substantially pure water or an aqueous solution that may also contain microorganisms, phosphorous compounds, carboxylate compounds such as acetate, proteins (e.g., yeasts), hydrogen release compounds, minerals, metal salts, and/or vitamins, among other components.

Specific examples of nutrient amendments may include carboxylic acids and salts thereof. They may also include cyclic and aromatic organic acids and salts thereof. They may further include sugars and sugar alcohols. They may yet further include alcohols, carboxyl and/or ketone-containing organic compounds. Still other nutrient compounds may include alkanes and polyaromatic compounds. Nutrient amendments may also include combinations of components, such as an amendment comprising a phosphorous compound, an acetate compound, and proteins (e.g., yeasts). Amendments may further include hydrogen release compounds. Additional examples of biological and chemical amendments that may be added into addition to the surfactants are described in co-assigned U.S. patent application Ser. No. 11/399,099 to Pfeiffer et al, filed Apr. 5, 2006, and titled “CHEMICAL AMENDMENTS FOR THE STIMULATION OF BIOGENIC GAS GENERATION IN DEPOSITS OF CARBONACEOUS MATERIAL” the entire contents of which is herein incorporated by reference for all purposes.

The surfactant may be provided to the formation in a single application or multiple applications spread out over time. The effects of the surfactant addition on the rate of methanogenesis may be monitored 106, for example by measuring recovery rates of gases and liquids from the formation. These may include the targeted metabolic products (e.g., hydrocarbons with enhanced hydrogen content, like methane) being stimulated by the surfactant addition. Monitoring may also include measurements of the partial pressures of gas phase metabolic products like methane, and measurements of molar concentrations of solution phase metabolic products. When the surfactant is added in two or more stages, this monitoring data may be used to tailor a subsequent surfactant addition to the formation conditions indicated by the data. For example, the data may be used to tailor the types, concentration, and absolute quantities of surfactants added to the formation, as well as additional components added with the surfactants. The metabolic products may also be recovered from the formation 108.

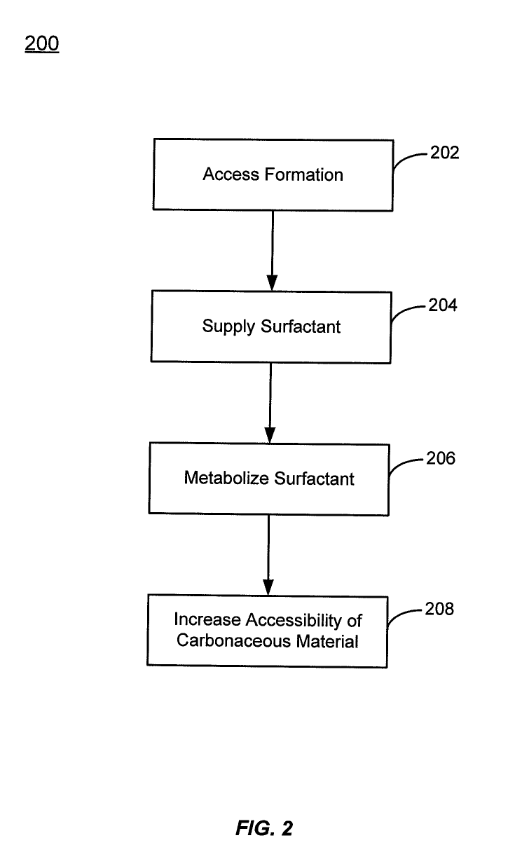

FIG. 2 shows selected steps in a method 200 of conditioning carbonaceous material for increased methanogenesis with a surfactant according to embodiments of the invention. The method 200 includes accessing a subterranean geologic formation though either a natural or man-made access point in the formation 202. The access point provides a route for a surfactant supplied from a source external to contact carbonaceous material in the formation 204.

The surfactant is selected such that at least some of the microorganisms in the consortium can utilize the surfactant as a nutrient source 206. In some instances, the surfactant may be metabolized by fermentative bacteria that are also active in the initial stages of methanogenesis metabolizing the carbonaceous material into more oxidized hydrocarbons such as organic acids and alcohols. Alternatively (or in addition) the surfactant may be metabolized by downstream microorganisms that convert the metabolic products of the fermentative bacteria into intermediate compounds and/or end-stage metabolic products with enhanced hydrogen content. These may include acetogenic bacteria that convert the organic acids and alcohols from the fermentative bacteria into simple carbon compounds such as acetate, carbon monoxide, carbon dioxide, etc., as well as non-carbon compounds like hydrogen (H₂). They may also include methanogens that convert acetate to methane and carbon dioxide via an acetate fermentation pathway, and/or convert hydrogen and carbon dioxide to methane and water via a carbonate reduction pathway. The surfactant may be selected for its ability to act as a nutrient source for one or more groups of these bacteria, and/or specific genera and species of bacteria in these groups.

Surfactants may be selected that can be wholly metabolized by a microorganism (e.g., smaller simpler surfactants) or may be partially metabolized by splitting, or breaking off a moiety that is wholly metabolized (e.g., larger, more complex surfactants). The metabolic products of the surfactant metabolism may be the same types of hydrocarbons having enhanced hydrogen content that are produced from the carbonaceous material, or different products. In many instances, microorganisms may more readily metabolize the surfactants than nearby carbonaceous material. The metabolizable surfactants provide a nutrient source that can be quickly utilized by the microorganisms, allowing their populations to grow at an accelerated rate at phase boundaries where the surfactants tend to concentrate. In some instances, the surfactants act like a seed material that helps provide a temporary nutrient supply until the microorganism consortium grows and adapts to using the carbonaceous material as its primary nutrient source.

In addition to providing nutrients, the surfactants may also use their more traditional properties as wetting agents, solubilizers, emulsifiers, dispersing agents, solvents, etc., to increase the accessibility of the carbonaceous material as a nutrient source for the microorganism consortium 208. Increasing the accessibility of the carbonaceous material may include moving a hydrocarbon trapped in a solid carbonaceous material (e.g., coal, shale, etc.) to a location where it can contact and be metabolized by a microorganism. The surfactant may facilitate the hydrocarbon being solubilized into a liquid phase, and/or transitioning from a less polar to a more polar liquid phase environment. The transported hydrocarbon may be smaller and less complex than the polymeric macromolecular structure that comprises the bulk of the carbonaceous material. These smaller hydrocarbons are often significantly easier for the microorganisms to metabolize than the complex macromolecules, and may represent a significant portion (if not the majority) of the carbonaceous material metabolized by the microorganisms.

Increasing the accessibility of the carbonaceous material may also include more widely distributing a polar aqueous-phase liquid containing microorganisms through the carbonaceous material. In this sense the wetting agent properties of the surfactant facilitates the spreading of the more polar liquid through a less polar carbonaceous material. The penetration and wetting of the carbonaceous material by the aqueous phase increases the surface area where the microorganisms and the carbonaceous material can make contact. The increased contact provides an increased supply of carbonaceous material that can be quickly metabolized by the microorganisms in the consortium. When a low concentration of these carbonaceous materials limits the rate of methanogenesis, the wetting properties of the surfactant helps alleviate this bottleneck by increasing the opportunities for carbonaceous components and microorganisms to make contact.

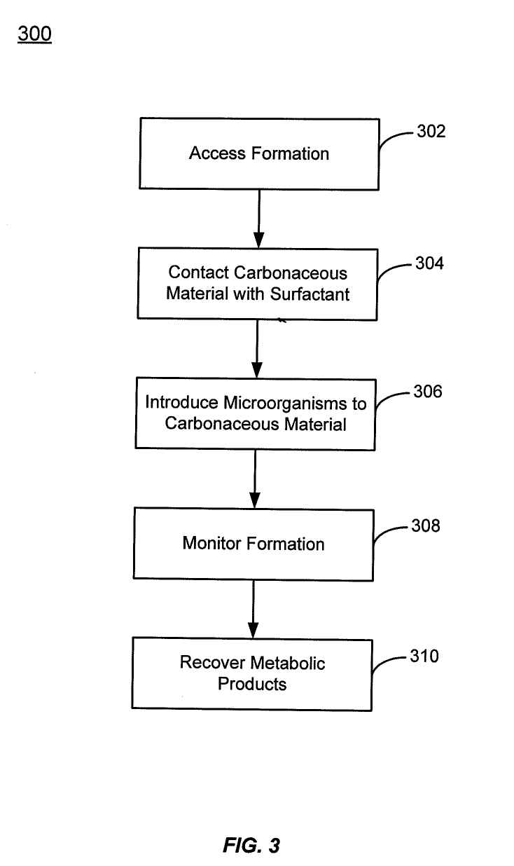

FIG. 3 is a flowchart showing selected steps in a method 300 of conditioning carbonaceous material according to additional embodiments of the invention. The method 300 may include the step of accessing a geologic formation 302, and contacting carbonaceous material in the formation with a surfactant 304. A period of time may then lapse before microorganism cells are introduced to at least a portion of the carbonaceous material contacted by the surfactant 306. The formation may be monitored for an increased rate of production of metabolic products from the biological decomposition of the carbonaceous material 308. One or more of these metabolic products may be recovered for applications, such as power generation (e.g., methane) 310.

Conditioning the carbonaceous material with the surfactant may help start methanogenesis in a previously inactive formation, as well as increase methanogenesis in a formation that is experiencing the biological production of gases such as methane. The surfactant may lower transportation barriers for materials migrating into and out of the carbonaceous material. In the case of carbonaceous materials with a significant solids component (e.g., coal, shale, tar sands, etc.), the surfactant may help extract highly metabolizable compounds (e.g., organic compounds containing 1-10 carbons) to locations in or on the surfaces of the material where microorganisms are present. The surfactants may also help introduce nutrients, activation compounds, enzymes, water, cells, etc., into the carbonaceous material.

There may be a conditioning period after the surfactant is introduced to the carbonaceous material that lasts from several hours to a month or more. Shorter periods may include about 1 hour, 2 hours, 3 hours, 4 hours, etc. Longer periods may include about 1 week, 2 weeks, 3 weeks, 4 weeks, etc. In some instances, the waiting period depends on the rate at which the surfactant can extract and/or introduce compounds from the carbonaceous material. In additional instance, the waiting period may depend on dilution and/or decomposition of the surfactant to a concentration that no longer inhibits growth of microorganisms in the consortium.

Following or concurrently during the conditioning period, a chemical and/or biological amendment(s) may be provided to the conditioned carbonaceous material. These amendments may include a group of microorganism cells transported in water. They may also include nutrient amendments that provide additional nutrients to a microorganism consortium present with the conditioned carbonaceous material.

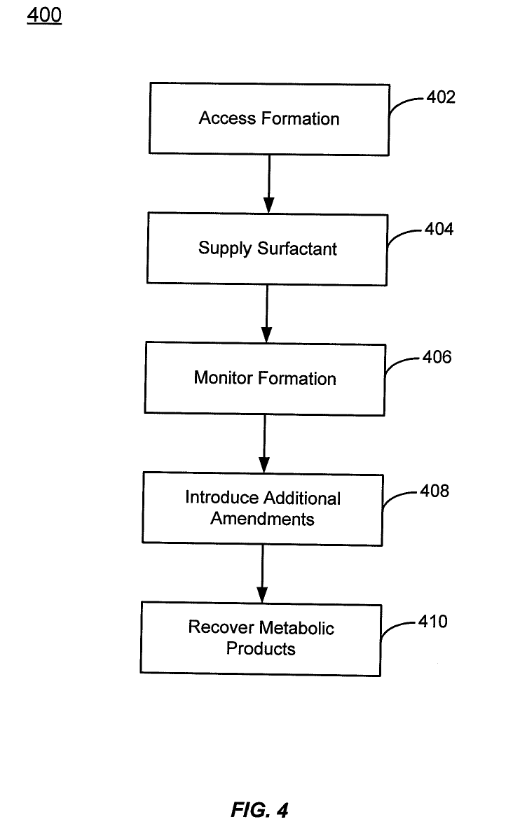

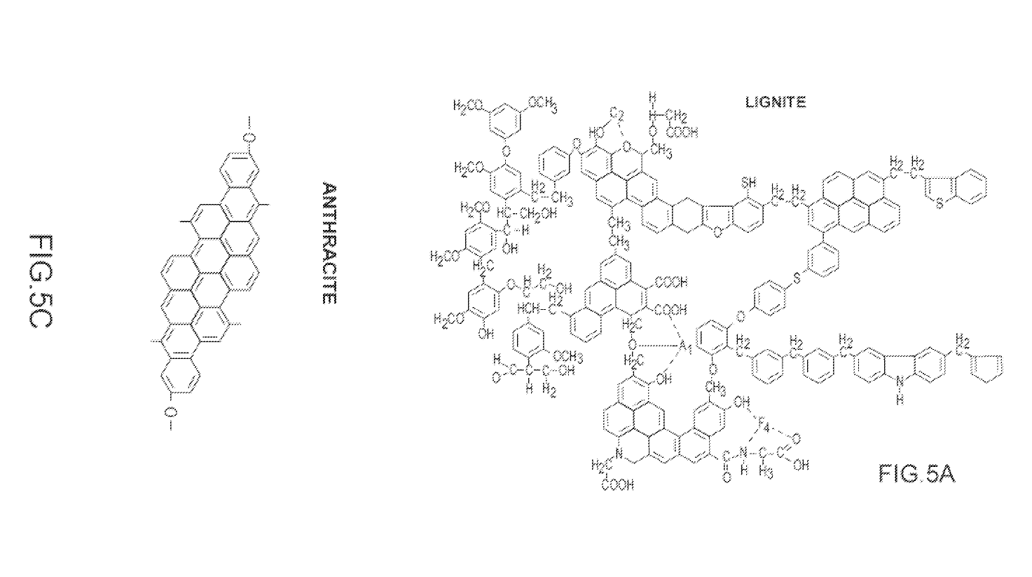

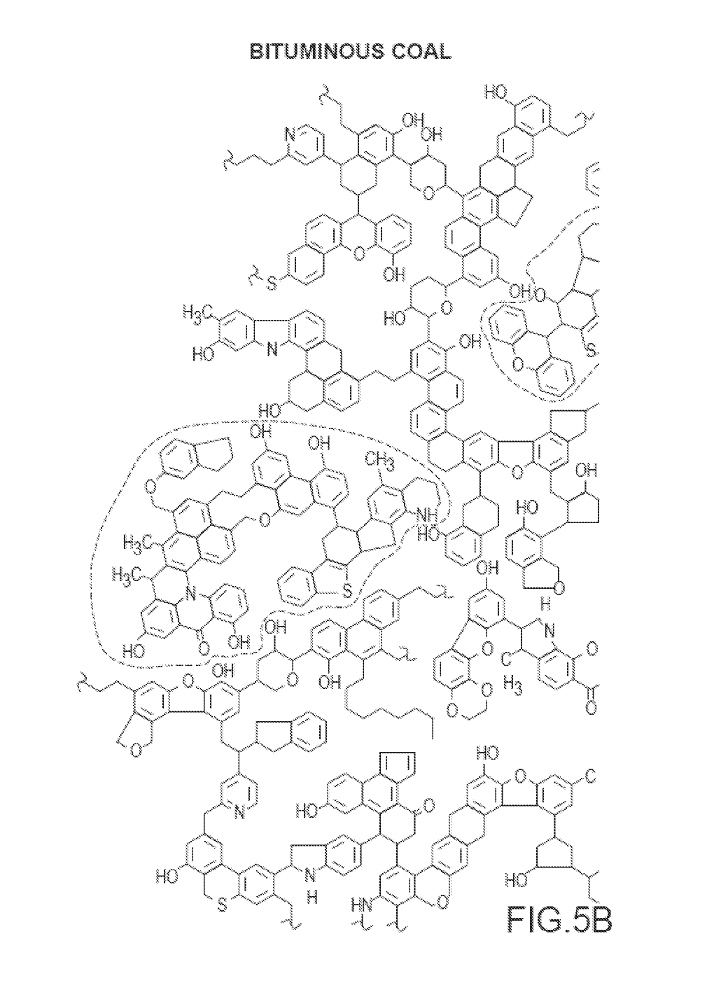

FIG. 4 is a flowchart showing selected steps in a method 400 of stimulating methanogenesis by providing a microorganism consortium with a surfactant composition according to embodiments of the invention. The method 400 may include the step of accessing a geologic formation 402, and supplying a surfactant composition 404 to a microorganism consortium in the formation. The method may further include monitoring the formation after the introduction of the surfactant composition 406 to determine if the surfactant is acting like a nutrient compound, an activation compound, or some combination of a nutrient and activation compound. When a surfactant is acting primarily or exclusively as a nutrient compound, then the increase in amount of metabolic products with enhanced hydrogen content may be stiochiometrically proportional to the amount of surfactant added. In contrast, when a surfactant is acting primarily as an activation compound, then the increased amount of metabolic products may be much larger than the amount of surfactant added.

A determination of whether the surfactant acts primarily as a nutrient or activation compound for the microorganism consortium can provide information for the introduction of additional amendments to the formation 408. For example, if the surfactant is acting primarily as a nutrient, then additional amendments may include larger quantities and/or concentrations of the surfactant than if it's acting primarily as a activation compound. In addition, a nutrient surfactant may require smaller quantities of additional nutrient compounds than an activation surfactant. The method may also include recovering metabolic products from the formation 410 for commercial applications such as transportation fuel, electrical power generation, etc.

The goal of the surfactant additions, whether acting as a food source, an activation agent, increasing the accessibility of a carbonaceous material, etc., is to increase the biogenic production of metabolic products with enhanced hydrogen content. These enhanced hydrogen content products have a higher mol. % of hydrogen atoms than the starting carbonaceous material. For example methane, which has four C—H bonds and no C—C bonds, has a higher mol. % hydrogen than a large aliphatic or aromatic hydrocarbon with a plurality of C—C single and double bonds. Additional details about compounds with enhanced hydrogen content may be found in co-assigned U.S. patent application Ser. No. 11/099,881, to Pfeiffer et al, filed Apr. 5, 2005, and entitled “GENERATION OF MATERIALS WITH ENHANCED HYDROGEN CONTENT FROM ANAEROBIC MICROBIAL CONSORTIA” the entire contents of which is herein incorporated by reference for all purposes.

### Exemplary Surfactants

As noted previously, surfactants (or surface acting agents) are compounds that are active at the interface between two phases, such as the interface between coal and water. Many surfactants are organic compounds that contain both hydrophilic groups and hydrophobic groups, making them amphiphilic (e.g., having both water-soluble and hydrocarbon-soluble components). Surfactants may also be classified by the ionic charge (or lack thereof) into four categories: 1) anionic (negatively charged), 2) cationic (positively charged), 3) non-ionic (no charge), and 4) zwitterionic (spatially separated positive and negative charge). They may also be classified as biodegradable or non-biodegradable. One or more of these categories of surfactants may be used in embodiments of the invention. Examples of anionic surfactants include Ninate 411, and Geopon T-77, among others. Examples of cationic surfactants include Benzalkonium Cl, among others. Examples of non-ionic surfactants include Tween 80, Tween 20, Triton X-100, Pluronic F68, Pluronic L64, Surfynol 465, Surfynol 485, Stilwet L7600, Rhodasurf ON-870, Cremophor EL, and Surfactant 10G, among others.

Surfactants may also be described according to their properties, which may include wetting, solubilizing other compounds, emulsifying, dispersion, and detergency, among other properties. Wetting reduces the surface tension of a liquid by reducing like attractions of molecules (e.g., polar water molecules) with one another and increasing the attraction towards an unlike compound (e.g., non-polar hydrocarbons). Surfactants with strong wetting ability increase the penetration and/or migration of aqueous solutions of microorganisms and/or chemical amendments into less polar carbonaceous materials, such as coal, oil, shale, etc. Surfactants known for their strong wetting properties include Triton X305, Surfactant 10G, Pluronic L64, Geropon T-77, Tetronic 1307, Surfynol 465, and Surfynol 485, among others.

Solubilizing refers to the ability of a surfactant to solubilize (e.g., dissolve) an otherwise insoluble material. In some instances, the insoluble material will be incorporated into micelles formed by the surfactant and distributed into the apparent solution. Micelles are spherical aggregates of a group of surfactant molecules that have their hydrophobic and hydrophilic groups radially arranged in particular directions. For example, micelles formed in water have their hydrophilic ends facing outwards to interact with the surrounding water molecules, and their hydrophobic tails facing inward to minimize contact with the water molecules. If the liquid media were non-polar (e.g., oil) the micelles would turn inside out, having their hydrophobic ends facing outward while the hydrophilic ends would face inwards and concentrate in the core of the aggregate. Micelles form when the surfactant concentration is high enough to reach a critical micelle concentration (CMC). As the micelles form, they can incorporate portions of the insoluble material into the micelle core and bring it into apparent solution. This allows water insoluble materials (e.g., hydrocarbons) to be solubilized in water, and oil insoluble materials (e.g., aqueous solutions) to be solubilized in oil.

Emulsification (emulsifying) refers to the ability of surfactants to form a stable emulsion from two or more immiscible liquids. For example, a surfactant with strong emulsification properties can form an emulsion of oil in an aqueous solution. Surfactants known for their strong emulsification properties include Triton X45, Ninate 411, Rhodasurf ON-870, Cremophor EL, and Tween surfactants, among others.

Dispersion refers to the ability of surfactants to keep insoluble particles in suspension by preventing them from aggregating with each other. As the size of the insoluble particles gets smaller, the dispersion formed by keeping them separated generally gets more stable. Surfactants known for their strong dispersion properties include Tetronic 1307, Geropon T-77, and Rhodasurf ON-870, among others.

Detergency refers to the ability of surfactants to remove materials and particles from a surface. Surfactants acting as detergents are used to release materials clinging or otherwise incorporated into a surface upon wetting. Surfactants known for their strong detergency properties include Bio-Terge AS-40, Standapol ES-1, Pluronic F68, and Chemal LA-9, among others.

As noted above, surfactants may be selected for their ability to provide a food source to microorganisms in addition to their more traditional surfactant properties. These may include surfactants that can be broken down into simple alkanes, alkenes, carboxylic acids, ketones, etc., which are precursors in the metabolic formation of acetate. The acetate may then be metabolized through the acetate fermentation pathway of the methanogenic microorganisms in the consortium into methane and carbon dioxide. The carbon dioxide may be converted into additional biogenic methane through the carbonate reduction pathway. Thus, this group of acetate producing surfactants not only provides a metabolic energy source for at least some of the microorganism consortium (including the methanogens), it also acts as a feedstock for useful metabolic products like methane.

Examples of these acetate producing surfactants may include 2-butoxyethanol, nonylphenol ethoxylate, Tween 20, Tween 80, and Triton X-100, among others. These surfactants share a common chemical moiety with Structure (1):

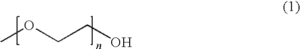

where n=1 to 20. For example, in the case of 2-butoxyethanol, n=1 and the leftmost oxygen is connected to a H₃C—CH₂—CH₂— group.

While not intending to be bound by any particular theory, it's believed that Structure (1) is a readily metabolizable moiety on the surfactant that can be further metabolized in one or more steps into acetate (i.e., CH₃COO—). The acetate may then be biogenically metabolized to methane as noted above.

### Exemplary Carbonaceous Materials

The surfactants may be used to treat a variety of carbonaceous materials. Typically, these carbonaceous materials are situated in subterranean geologic formations that have formed the carbonaceous material from decomposed organic matter over the course of thousands to millions of years (e.g., so-called fossil fuels). Examples of carbonaceous materials may include bituminous coal, subbituminous coal, anthracite, oil, carbonaceous shale, oil shale, tar sands, tar, lignite, kerogen, bitumen, and peat, among other carbonaceous materials.

The surfactants may be applied to solid carbonaceous materials to make components of the material more accessible to a microorganism consortium. Coal for example, includes large, complex macromolecules such as subbituminous coal, as well as smaller simpler organic molecules such as small polar-organic molecules like alcohols, ketones, aldehydes, ethers, esters, and organic acids, monoaromatic compounds, simple polyaromatic compounds (e.g., 2-3 ring polyaromatic compounds), and short-chained alkanes, alkenes, and alkynes, among other small and intermediate sized organic molecules.

One conventional classification for coal is coal rank. Coals of increasing rank generally have more densely packed aromatic rings (i.e., the number of aromatic rings per macromolecular “unit” increases) and are generally more dense and harder than lower ranked coals. Coals of increasing rank include lignite, subbituminous, volatile bituminous, bituminous coals that increasingly consist of anthracite. Representative macromolecular structures of lignite, anthracite, and bituminous coal are shown in FIGS. 4A-C, respectively although there can be significant variation in the actual structures. These macromolecules commonly have molecular weights well in excess 1,000 g/mol, and commonly in excess of 1,000,000 g/mol. There is also evidence that fragments (e.g., 400-1000 g/mol) of a larger macromolecule supports methanogenesis.

One use of surfactants is to move the smaller and intermediate sized molecules contained in the macromolecular coal structure to locations that are accessible to the microorganism consortium. Evidence suggests that if even a small fraction of these molecules are metabolized by the consortium, they could provide significant quantities of useful biogenic gases such as methane. For example, Table 1 below shows the quantities of selected classes of organic compounds extracted from a sample of coal with methylene chloride (MeCl) and methanol (MeOH). The Table also lists the equivalents of methane these extracted compounds represent.

TABLE 1
Theoretical Methane Yields From Compounds
Extracted from Coal Sample

| | Quantity in mg/g | ~Theoretical |
|---|---|---|
| Compound Class | coal | CH₄ Yield |
| Asphaltenes | 31.8 | 1,528 |
| Saturates | 1.8 | 99 |
| Aromatics | 4.1 | 198 |
| n-alkanes | 0.05 | 2.9 |
| Polars | 7.3 | 289 |
| C14-C30 alkanoic acids | 0.02 | 0.8 |
| Acetate | 0.11 | 1.8 |
| Total Extractable Compounds | 46.1 | 2,163 |
| Non-Extractable Hydrocarbons | 703.9 | 17,764 |

Asphaltenes are intermediate-sized aromatic clusters (˜2-6 rings) with aliphatic side chains and/or bridges. Average molecular weight for these compounds is about 500-1000 g/mol. Asphaltenes are known to be biodegradable under aerobic conditions, and may also be metabolizable (in whole or part) by an anaerobic microorganism consortium. Additional examples of extractable compounds may include acetates, formates, oxalates, pthalates, benzoates, phenols, cresols, n-alkanes, branched alkanes, cyclic alkanes, monoaromatic organic compounds, 2 and 3 membered ring polyaromatic organic compounds (e.g., naphthalenes, phenanthrenes, etc.). These compounds and classes of compounds, alone or in combination, may be metabolized by members of a methanogenic microorganism consortium into metabolic products with enhanced hydrogen content.

### Exemplary Consortium Organization and Microorganism Genera

The microorganism consortium that converts the carbonaceous material into metabolic products with enhanced hydrogen content may be made up of made up of 10 or more, 20 or more, 30 or more different species of microorganisms. Thus, it should be appreciated that the conversion of one metabolite to another may involve a plurality of microorganisms using a plurality of metabolic pathways to metabolize a plurality of intermediate compounds.

The microorganism consortium may be made up of one or more subpopulations of microorganisms, where each consortium subpopulation may be identified by the role it plays in the overall conversion of starting carbonaceous materials to metabolic end products. Each subpopulation may include a plurality of microorganisms that may belong to the same or different genera, and belong to the same or different species. When a subpopulation includes a plurality of different species, individual species may work independently or in concert to carry out the metabolic function of the subpopulation. The term microorganism as used here includes bacteria, archaea, fungi, yeasts, molds, and other classifications of microorganisms. Some microorganism consortiums can have characteristics from more than one classification (such as bacteria, archea, etc.).

Because subterranean formation environments typically contain less free atmospheric oxygen (e.g., O₂) than found in tropospheric air, the microorganisms are described as anaerobic microorganisms. These microorganisms can live and grow in an atmosphere having less free oxygen than tropospheric air (e.g., less than about 18% free oxygen by mol.). In some instances, the anaerobic microorganisms operate in a low oxygen atmosphere, where the O₂ concentration is less than about 10% by mol., or less than about 5% by mol., or less than about 2% by mol., or less than about 0.5% by mol. Water present in the formation may also contain less dissolved oxygen than what is typically measured for surface water (e.g., about 16 mg/L of dissolved oxygen). For example, the formation water may contain about 1 mg/L or less of dissolved oxygen.

The microorganisms that make up the consortium may include obligate anaerobes that cannot survive in an atmosphere with molecular oxygen concentrations that approach those found in tropospheric air (e.g., 18% to 21%, by mol. in dry air) or those for which oxygen is toxic. The consortium may also include facultative aerobes and anaerobes that can adapt to both aerobic and anaerobic conditions. A facultative anaerobe is one which can grow in the presence or absence of oxygen, but grow better in the presence of oxygen. A consortium can also include one or more microaerophiles that are viable under reduced oxygen conditions, even if they prefer or require some oxygen. Some microaerophiles proliferate under conditions of increased carbon dioxide of about 10% mol or more (or above about 375 ppm). Microaerophiles include at least some species of Spirillum, Borrelia, Helicobacter and Campylobacter.

In some embodiments, the ratio of aerobes to anaerobes in a consortium may change over time. For example, a consortium may start in an environment like oxygenated water before being introduced into a sub-surface anaerobic formation environment. Such a consortium starts out with higher percentages of aerobic microorganisms and/or facultative anaerobes to metabolize carbonaceous materials in the formation. As the free oxygen concentration decreases, the growth of the aerobes is slowed, and growing anaerobic microorganisms or consortiums metabolize the metabolic products of the aerobic microorganisms into organic compounds with higher mol. % of hydrogen atoms.

Consortium embodiments may be described by dividing the consortia into three or more consortia defined by the function they play in the conversion of starting hydrocarbons in native carbonaceous materials (like coal, shale, and oil) into end hydrocarbons like methane. The first microbial subpopulation may include one or more microorganisms that break down the starting hydrocarbons into one or more intermediate organic compounds. For example, when the carbonaceous material is bituminous coal, one or more microorganisms of the first subpopulation may split an alkyl group, or aromatic hydrocarbon from the polymeric hydrocarbon substrate. This process may be referred to as the metabolizing of the carbonaceous material, whereby the complex macromolecular compounds found in the carbonaceous material are decomposed into lower molecular weight hydrocarbon residues.

The second microbial subpopulation may include one or more microorganisms that metabolize or otherwise transform the intermediate organic compounds into other intermediate organic compounds, including compounds with oxidized, or more highly oxidized, carbons (e.g., alcohol, aldehyde, ketone, organic acid, carbon dioxide, etc.). These second stage intermediate organics are typically smaller, and may have higher mol. % of hydrogen atoms, than the starting organic compounds, with one or more carbons being split off as an oxidized carbon compound. “Oxidized carbon” refers to the state of oxidation about a carbon atom wherein an order of increasingly oxidized carbon atoms is from —C—H (carbon bonded to hydrogen); to —C—OH (carbon bonded to a hydroxyl group, such as an alcohol as a non-limiting example); —C═O (carbon double-bonded to oxygen); —COOH (carbon as part of a carboxyl group); and CO₂ (carbon double-bonded to two oxygen atoms) which is the most oxidized form of carbon. As a carbon atom is more oxidized, the total energy associated with the bonds about that atom decreases. This is consistent with the general tendency that as microorganisms extract energy from carbon containing molecules, they remove hydrogen atoms and introduce oxygen atoms. As used herein, “oxidized carbon” does not include any carbon atom that is only bonded to hydrogen and/or one or more carbon atoms.

Because carbon dioxide is generally considered to contain no obtainable energy through oxidation, the present invention is based in part on the advantageous use of microorganisms to convert the carbon atom in carbon dioxide into a higher energy state (i. e., a more reduced state), such as in methane. This may be considered a reversal of the oxidation process that produced carbon dioxide by members of a consortium of the invention.

The third microbial consortium subpopulation includes one or more microorganisms that metabolize the final intermediate organic compounds into at least one smaller hydrocarbon (having a larger mol. % hydrogen than the intermediate hydrocarbon) and water. For example, the final intermediate compound may be acetate (H₃CCOO⁻) that is metabolized by members of the third consortium into methane and water. In other examples, a third consortium may metabolize the acetate into methane and carbon dioxide via the process of acetoclastic methanogenesis. A consortium according to these embodiments may include at least one consortium of microorganisms that does not form methane by the pathway of reducing carbon dioxide to methane.

In other embodiments, a consortium may include one or more subpopulations having different functions than those described above. For example, a consortium may include a first subpopulation that breaks down the starting hydrocarbons in the carbonaceous material into one or more intermediate organic compounds, as described above. The second subpopulation, however, metabolizes the intermediate organics into carbon dioxide and molecular hydrogen (H₂). A third subpopulation of the consortium, which includes one or more methanogens, may convert CO₂ and H₂ into methane and water.

A consortium may include intra-subgroup and inter-subgroup syntrophic interactions. For example, members of the second and third subgroup above may form a syntrophic acetate oxidation pathway, where acetate is converted to methane at an enhanced metabolic rate. Microorganisms in the second subgroup convert acetic acid and/or acetate (H₃CCOO⁻) into carbon dioxide and hydrogen, which may be rapidly metabolized by methanogens in the third subgroup into methane and water. Removal of second subgroup metabolites (e.g., hydrogen, carbon dioxide) by members of the third subgroup prevents these metabolites from building up to a point where they can reduce metabolism and growth in the second subgroup of the consortium. In turn, the second subgroup provides a steady supply of starting materials, or nutrients, to members of the third subgroup. This syntrophic interaction between the subgroups results in the metabolic pathway that converts acetate into methane and water being favored by the consortium.

Thus as used herein, syntrophy refers to symbiotic cooperation between two metabolically different types of microorganisms (partners) wherein they rely upon each other for degradation of a certain substrate. This often occurs through transfer of one or more metabolic intermediate(s) between the partners. For efficient cooperation, the concentration of the metabolic intermediate(s) may be kept low. In one non-limiting example pertinent to the present invention, syntrophs include those organisms which oxidize fermentation products, such as propionate and butyrate, from upstream consortium members. These organisms require low concentrations of molecular hydrogen to ferment substrates to acetate and carbon dioxide, so are symbiotic with methanogens, which help maintain low molecular hydrogen levels.

Genera of microorganisms included in the consortium may include, Thermotoga, Pseudomonas, Gelria, Clostridia, Moorella, Acetobacterium, Sedimentibacter, Acetivibrio, Syntrophomonas, Spirochaeta, Treponema, Thermoacetogenium, Bacillus, Geobacillus, Pseudomonas, Sphingomonas, Methanobacter, Methanosarcina, Methanocorpusculum, Methanobrevibacter, Methanothermobacter, Methanolobus, Methanohalophilus, Methanococcoides, Methanosalsus, Methanosphaera, Methanoculleus, Methanospirillum, Methanocalculus, Methanosaeta, Granulicatella, Acinetobacter, Fervidobacterium, Anaerobaculum, Ralstonia, Sulfurospirullum, Acidovorax, Rikenella, Thermoanaeromonas, Desulfovibrio, Desulfomicrobium, Desulfobulbus, Desulfobacter, Desulfosporosinus, Dechloromonas, Acetogenium, Bacteroides, Desulfuromonas, Pelobacter, Geobacter, Syntrophobacter, Syntrophus, Propionibacterium, Ferribacter, Fusibacter, Thiobacillus, Campylobacter, Sulfurospirillum, Thauera, Rhodoferax, and Arcobacter, among others. Additional descriptions of microorganisms that may be present can be found in commonly assigned U.S. patent application Ser. No. 11/099,881, filed Apr. 5, 2005, and titled “Generation of materials with Enhanced Hydrogen Content from Anaerobic Microbial Consortia”; U.S. patent application Ser. No. 11/099,880, also filed Apr. 5, 2005, titled “Generation of Materials with Enhanced Hydrogen Content from Microbial Consortia Including Thermotoga”; and U.S. patent application Ser. No. 11/971,075, filed Jan. 8, 2008, ant titled “Generation of Materials with Enhanced Hydrogen Content from Anaerobic Microbial Consortia Including Desulfuromonas or Clostridia” the entire contents of all three applications hereby being incorporated by reference for all purposes.

### EXPERIMENTAL

Experiments were conducted to compare biogenic methane generation from coal samples after introducing an amendment of a surfactant. For each experiment, methane generation from coal samples from the Powder River Basin in Wyoming and shale samples from the Antrim Shale in Michigan was periodically measured over the course of more than 100 days. Each 2.5 gram coal sample or 5 g shale sample was placed in a 30 ml serum bottle with 15 mL of water that was also taken from the formation. The coal or shale and formation water were placed in the serum bottle while working in an anaerobic glove bag. The headspace in the bottle above the sample was flushed with a mixture of N₂ and CO₂ (95/5).

Amendments were then added to the samples. Surfactants were tested at concentrations of 0.05 to 0.5 g/L. Surfactants were tested alone and in combination with other amendments, including proteins (e.g., yeast extract), phosphate and acetate. The samples were then sealed, removed from the glove bag, and stored at a temperature close to the in situ temperature for the coal or shale samples over the course of the experiments.

The methane levels in the headspace above the samples was periodically measured and recorded. The methane was measured by running samples of the headspace gases through a gas chromatograph equipped with a thermal conductivity detector. The highest levels of methane production in coal containing bottles after more than 100 days occurred in samples treated with an amendment of the following surfactants: 2-butoxyethanol, Benzalkonium chloride, Geropon T-77, Pluronic F68, Pluronic L64, Simple Green, Stilwet L7600, Surfactant 10G, Surfynol 465 and Tetronic 1307. The highest levels of methane production in shale containing bottles after more than 100 days occurred in samples treated with an amendment of the following surfactants: 2-butoxyethanol, Rhodasurf ON-870, Simple Green, and Surfynol 485. Other surfactants tested also showed increased methane production over that in control bottles.

The combination of surfactant amendments with yeast extract and phosphate gave the most methane production in bottles. These additional nutrients provide better growth conditions for hydrocarbon degrading consortium members.

Surfactant amendments were converted to intermediates, including short chain carboxylic acids, prior to conversion to methane. This suggests that microbial consortia present in coal and shale and associated waters have the capability to use surfactants as nutrients in addition to their hydrocarbon substrates.

The methane produced in the experiments described here is believed to come from a combination of surfactant amendment and hydrocarbons in coal and shale. The stimulatory effect of the surfactant amendment is not limited to enhancing the conversion of the added surfactant to methane. It also includes stimulating the microorganisms to use methanogenic metabolic pathways that convert the coal substrate into methane.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the surfactant” includes reference to one or more surfactants and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. A method of increasing biogenic production of a metabolic product with enhanced hydrogen content, the method comprising:
accessing a subterranean geologic formation that includes a carbonaceous material;
providing a surfactant solution to the geologic formation, wherein the surfactant solution increases a rate at which the metabolic product is biogenically produced in the geologic formation.

2. The method of claim 1, wherein the surfactant solution comprises an alkoxyethanol.

3. The method of claim 2, wherein the alkoxyethanol comprises 2-butoxyethanol.

4. The method of claim 1, wherein the carbonaceous material comprises coal or shale.

5. The method of claim 1, wherein the metabolic product is methane.

6. A method of conditioning a carbonaceous material in a subterranean geologic formation for metabolism into a compound with enhanced hydrogen content by a microorganism consortium, the method comprising:
accessing the subterranean geologic formation through an access point;
contacting the carbonaceous material with a surfactant;
allowing the microorganism consortium to utilize the surfactant as a first nutrient source; and
increasing accessibility of the carbonaceous material as a second nutrient source for the microorganism consortium with the surfactant, wherein the microorganism consortium metabolizes the carbonaceous material into the compound with the enhanced hydrogen content.

7. The method according to claim 6, wherein the surfactant comprises an alkoxyethanol.

8. The method according to claim 7, wherein the alkoxyethanol comprises 2-butoxyethanol.

9. The method according to claim 6, wherein the microorganism consortium metabolizes at least a portion of the surfactant into an acetate compound.

10. The method according to claim 6, wherein increasing the accessibility of the carbonaceous material as the second nutrient source for the microorganism consortium comprises moving a hydrocarbon from the carbonaceous material into contact with the microorganism.

11. The method of claim 6, wherein increasing the accessibility of the carbonaceous material as the second nutrient source for the microorganism consortium comprises increasing contact between the microorganism consortium and the carbonaceous material.

12. The method of claim 6, wherein increasing the accessibility of the carbonaceous material as the second nutrient source for the microorganism consortium comprises converting a portion of the carbonaceous material from a solid phase into a solution phase.

13. The method according to claim 6, wherein the carbonaceous material comprises coal or shale.

14. The method of claim 6, wherein the compound with enhanced hydrogen content comprises methane.

15. A method of increasing the accessibility of a carbonaceous material in a subterranean geologic formation to a microorganism consortium, the method comprising:
accessing the subterranean geologic formation;
contacting the carbonaceous material with a surfactant, wherein the surfactant moves a first hydrocarbon from the carbonaceous material into contact with the microorganism consortium; and
having the microorganism consortium metabolize the first hydrocarbon into a metabolic product with enhanced hydrogen content compared with the first hydrocarbon species.

16. The method of claim 15, wherein the first hydrocarbon comprises an alkane or a monoaromatic compound.

17. The method of claim 16, wherein the first hydrocarbon comprises a phenol.

18. The method of claim 15, wherein the surfactant comprises 2-butoxyethanol.

19. The method of claim 15, wherein the method comprises having the microorganism consortium metabolize a second hydrocarbon from the carbonaceous material that has not been moved by the surfactant.

20. The method of claim 19, wherein the carbonaceous material comprises coal or shale.

21. The method of claim 20, wherein the second hydrocarbon comprises a portion of a macromolecule in the coal.

22. The method of claim 15, wherein the metabolic product with enhanced hydrogen content comprises methane.
